(19) United States
(12) Patent Application Publication
(10) Pub. No.: US 2017/0168005 A1
(43) Pub. Date: Jun. 15, 2017

(54) SYSTEMS FOR ACTIVATING HANDHELD ELECTRONIC DEVICES

(71) Applicant: BroadMaster Biotech Corp., Taoyuan City (TW)

(72) Inventor: YI-LUNG CHEN, Taoyuan City (TW)

(21) Appl. No.: 15/367,068

(22) Filed: Dec. 1, 2016

(30) Foreign Application Priority Data
Dec. 9, 2015 (TW) 104141374

(51) Int. Cl. G01N 27/327; G01N 33/49

(52) U.S. Cl. CPC G01N 27/3273; G01N 33/49; G01N 27/3272

(57) ABSTRACT

Systems for activating glucometers are provided. The systems comprise a strip and a glucometer. Furthermore, the strip comprises at least two pins configured at different locations from the bottom edge of the strip. Once the strip is placed into the glucometer, the two pins electrically connect to at least two pads configured in the glucometer and lead to a short circuit between the two pads. The short circuit is identified as an activation signal to activate the glucometer. Accordingly, the pins provide a security mechanism to ensure that the glucometer is activated when electrical connections are properly accomplished.

CROSS-REFERENCE TO RELATED APPLICATION AND CLAIM OF PRIORITY

This application claims the benefit of Taiwanese Patent Application No. 104141374, filed on Dec. 9, 2015, in the Taiwan Intellectual Property Office, the disclosure of which is incorporated herein in its entirety by reference.

1. Technical Field

At least one embodiment of the prevent invention is a system for activating handheld electronic devices. More particularly, a system for activating glucometers which is to suppress the premature activation of a glucometer prior to that the strip is in position.

2. Description of the Related Art

Glucometers can be roughly classified into photochemical or electrochemical categories. The photochemical glucometers utilize colorimeters to detect blood glucose levels. After a blood sample is developed, a colorimeter then provides a beam passing through the blood sample to a photoelectric cell to obtain readings from the photoelectric cell, in which the readings reflect blood glucose levels in the blood sample. In contrast, the electrochemical glucometers utilize multiple electrodes to detect blood glucose levels. After a blood sample is treated with enzymes and generates electrons, the glucometer then infers blood glucose levels from the quantity of the electrons or the magnitude of electric current detected by the electrodes.

Blood glucose levels are in relationships with several chronic diseases. Therapies to these chronic diseases are usually accompanied by regular monitoring and recording of blood glucose levels in a long term. Glucometer manufacturers have developed a series of point-of-care (POC) products. A POC product contains biosensors, microprocessors, and related units in one single mobile device. The POC products largely simplify detection processes and therefore bring the idea of self-monitoring to real. With automation-based designs, a user may simply insert a strip to activate the automatic detection process of a glucometer to obtain and record readings of blood glucose levels.

However, the automation-based designs of known glucometers have some defects. The POC products are usually operated by non-medical specialists. The operations of these POC products are easily affected by mishandling and therefore result in detection errors. For example, the glucometer without a fine activation mechanism usually provide poor reading results since the glucometer tends to be prematurely activate by strips. Accordingly, systems for activating handheld electronic devices are needed in the art to mitigate the premature activation of glucometers by strips before the strips are in position.

### SUMMARY

At least one embodiment of the prevent invention is a system for activating handheld electronic devices. More particularly, a system for activating glucometers which is to suppress the premature activation of a glucometer prior to that the strip is in position.

At least one embodiment of the present invention is a system for activating glucometers comprising a strip and a glucometer. The strip comprises a substrate and a first electrode, in which the substrate contains a sample end and a connection end and the first electrode contains a first pin and a second pin connecting with each other. More specifically, the connection end has a bottom edge. The first pin is extending to the bottom edge, and the second pin is extending to a place closer to the inner as compared to the first pin. That is, the second pin is a first distance away from the bottom edge as compared to the first pin.

As for the glucometer, the glucometer comprises a slot. The slot is configured to receive the connection end of the strip, in which the slot itself contains a first contact site and a second contact site configured to electrically connect with the first pin and the second pin on the strip respectively. Moreover, the glucometer comprises a sensor electrically connecting with the slot. Specifically, the sensor is configured to activate the glucometer once a predetermine event is detected. The predetermined event is that the first pin and the second pin are electrically connecting to the first contact site and the second contact site respectively

At least one embodiment of the present invention utilizes a second pin extending a shorter distance to ensure that the second pin will not contact with the second contact site unless the strip moves further into the slot. This mechanism can mitigate the errors resulted from the premature activation of a glucometer prior to that the strip is in position.

### BRIEF DESCRIPTION OF THE DRAWINGS

FIGS. 1A-1C are schematic views illustrating a system for activating glucometers, in accordance with some references.

FIGS. 2A-2C are schematic views illustrating a system for activating glucometers, in accordance with some references.

FIG. 3 is an exploded view illustrating a strip, in accordance with one embodiment of the present invention.

FIG. 4 is a schematic view illustrating a strip, in accordance with one embodiment of the present invention.

FIG. 5 is a schematic view illustrating a glucometer, in accordance with one embodiment of the present invention.

FIGS. 6A-6C are schematic views illustrating a system for activating glucometers, in accordance with one embodiment of the present invention.

FIGS. 7A-7C are schematic views illustrating a system for activating glucometers, in accordance with one embodiment of the present invention.

### DESCRIPTION OF THE PREFERRED EMBODIMENTS

At least one embodiment of the prevent invention is a system for activating glucometer. More particularly, a system for activating glucometers which is used to prevent premature activation of a glucometer prior to that the strip is in position.

The system for activating glucometers comprises a strip and a glucometer. The strip comprises a substrate and a first electrode, in which the substrate further contains a sample end and a connection end while the first electrode further contains a first pin and a second pin connecting with each other. Moreover, the connection end has a bottom edge. The first pin is extending to the bottom edge, and the second pin is extending to a place closer to the inner as compared to the first pin. That is, the second pin is a distance away from the bottom edge as compared to the first pin.

The glucometer, on the other hand, comprises a slot. The slot is configured to engage with the connection end of the strip, in which the slot itself contains a first contact site and a second contact site configured to electrically connect with the first pin and the second pin respectively. Moreover, the glucometer comprises a sensor electrically connecting with the slot. The sensor is configured to activate the glucometer once a predetermine event is detected. More specifically, the predetermined event is that the first pin and the second pin are electrically connecting to the first contact site and the second contact site respectively, which therefore results in that the first contact site and the second contact site are electrically connected.

In the present embodiment, the term “activate” may refer to a transition of the glucometer from a low-power state to a high-power state. For example, the glucometer may transit from the sleeping mode to the working mode or the glucometer may supply power to more internal components.

In the present embodiment, the term “inward” may refer to the direction from a reference point toward the center of an object that reference point is attaching on. For example, “a pin is extending to a place a distance inward from the bottom edge” refer to that the pin is extending toward the bottom edge but that extension stops at a place which is a distance away from the bottom edge.

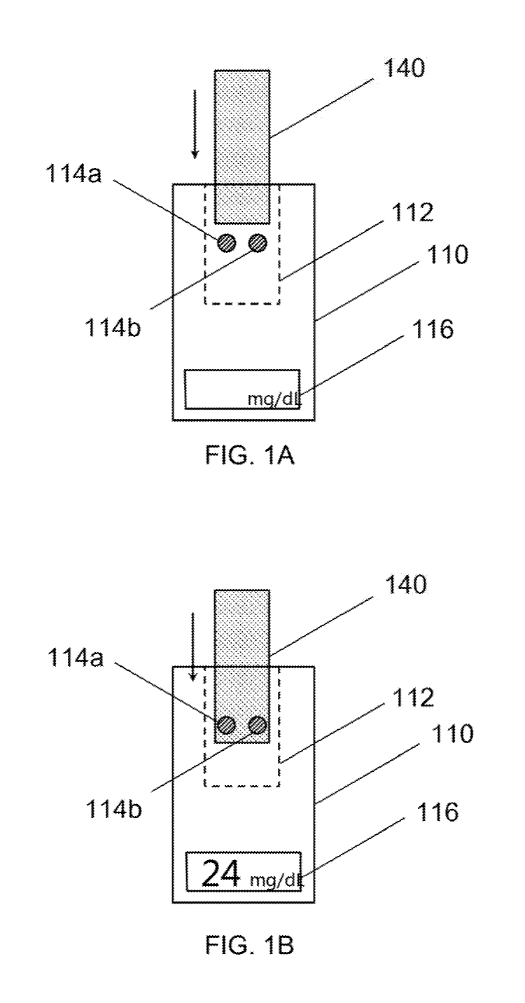

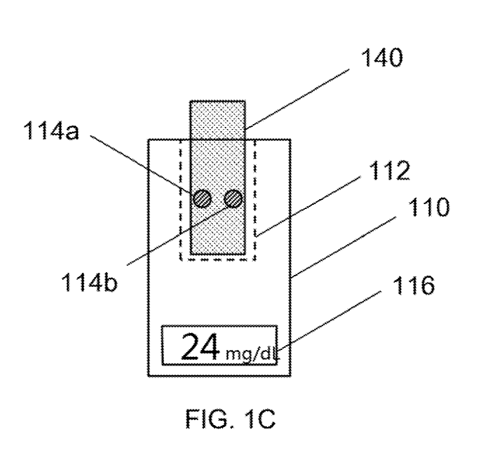

FIGS. 1A-1C are schematic views illustrating a system for activating glucometers, in accordance with some references. In that references, the system for activating glucometers comprises a glucometer 110 and a first electrode 140 disposed on a strip (not shown). The glucometer 110 further comprises a slot 112, a first connection site 114a, a second connection site 114b, and a display 116. If the strip is inserted into the slot 112, the first electrode 140 and both the first connection site 114a and the second connection site 114b are electrically connected together and therefore activates the glucometer 110.

More particularly, FIGS. 1A-1C illustrate a system for activating glucometers embodied by a known glucometer 100 and the process of inserting a strip into the glucometer 110 for activation. FIG. 1A illustrates the early stage of the process that inserting the strip into the glucometer 110. In FIG. 1A, the first electrode 140 is separated from both the first contact site 114a and the second contact site 114b which are disposed in parallel. The glucometer 110 in FIG. 1A is therefore not activated and the display 116 does not present the blood glucose reading. FIG. 1B illustrates the middle stage of the process, in which the strip is not fully inserted into the slot 112. In FIG. 1B, the first electrode 140 is contacting with the first contact site 114a and the second contact site 114b which are disposed in parallel. The glucometer 110 in FIG. 1B is therefore activated and the display 116 is presenting the blood glucose reading. FIG. 1C illustrates the late stage of the process, in which the strip is fully inserted into the slot 112. In FIG. 1C, the first electrode 140 is contacting with the first contact site 114a and the second contact site 114b which are disposed in parallel. However, the glucometer 110 has been activated and the detection of blood glucose level has completed in the middle stage. That is, the glucometer 110 has a defect that the detection is completed before the strip has been fully in position.

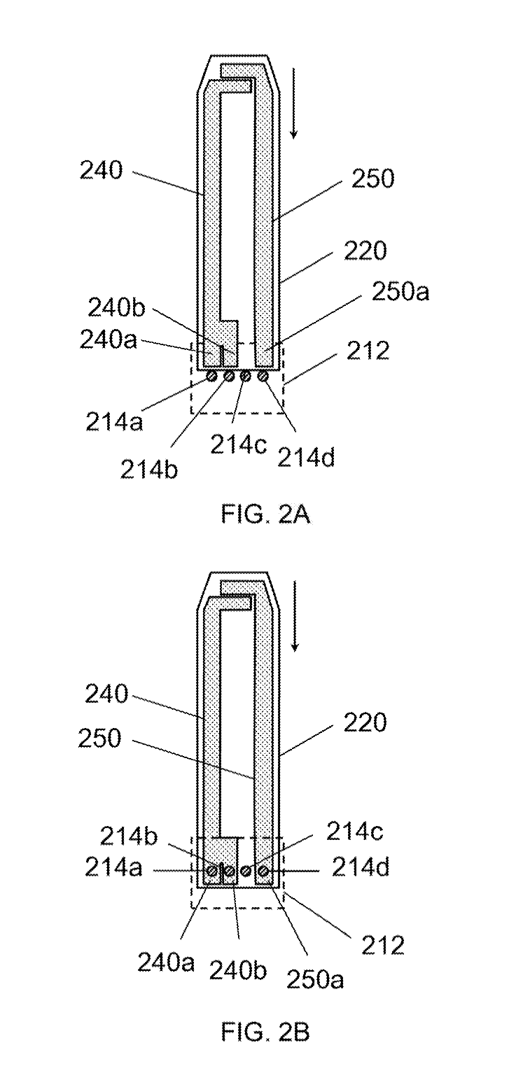

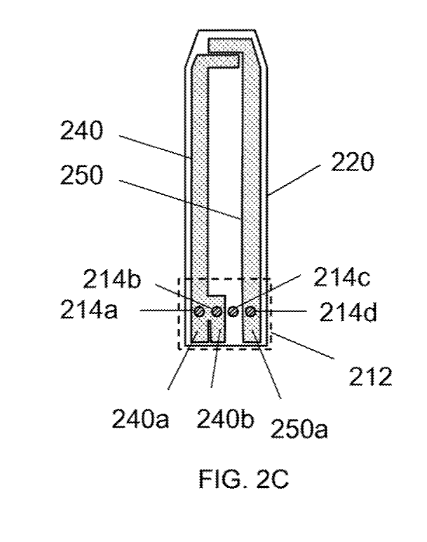

FIGS. 2A-2C are schematic views illustrating a system for activating glucometers, in accordance with some references. In that references, the system for activating glucometers comprises a slot 212 configured in a glucometer (not shown) and a strip. The strip comprises a substrate 220, a first electrode 240, and a second electrode 250, in which the first electrode 240 and the second electrode are disposed on substrate 220. The slot comprises a first contact site 214a, a second contact site 214b, a third contact site 214c, and a fourth contact site 214d which are disposed in parallel. A first pin 240a and a second pin 240b protruding from the first electrode 240 are configured to be electrically connected with the first contact site 214a and the second contact site 214b when the strip is inserted into the slot 212, in which the electrical connection will result in the activation of the glucometer. In contrast, the third pin 250a extending from the second electrode 250 is configured to be electrically connected with the fourth contact site 214d.

More particularly, FIGS. 2A-2C illustrate a system for activating glucometers embodied by a known glucometer and the process of inserting a strip into the slot 212 for activation. FIG. 2A illustrates the early stage of the process that inserting the strip into the slot 212. In FIG. 2A, the first pin 240a, the second pin 240b, and the third pin 250a are separated from the first contact site 214a, the second contact site 214b, and the fourth contact site 214d which are disposed in parallel. The glucometer 110 in FIG. 1A is therefore not activated. FIG. 2B illustrates the middle stage of the process, in which the strip is not fully inserted into the slot 212. In FIG. 1B, the first pin 240a, the second pin 240b, and the third pin 250a are contacting with either the first contact site 214a, the second contact site 214b, or the fourth contact site 214d which are disposed in parallel. The glucometer is therefore activated and the detection of blood glucose level is completed. FIG. 2C illustrates the late stage of the process, in which the strip is firmly inserted into the slot 212. In FIG. 2C, the first pin 240a, the second pin 240b, and the third pin 250a are contacting with the first contact site 114a, the second contact site 114b, and the fourth contact site 114d which are disposed in parallel respectively. However, the glucometer has been activated and the detection of blood glucose level has completed in the middle stage. That is, the glucometer has a defect that the detection is completed before the strip has been fully in position.

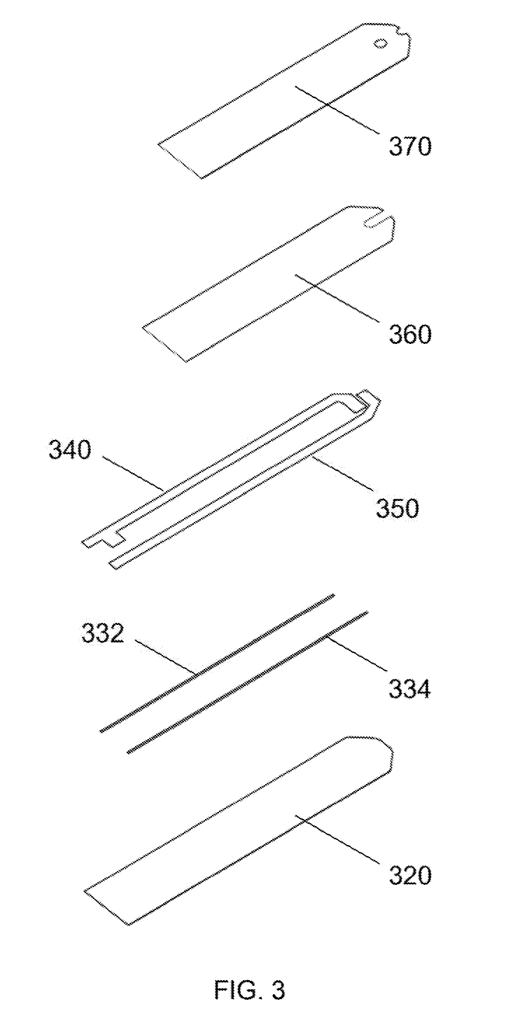

FIG. 3 is an exploded view illustrating a strip, in accordance with one embodiment of the present invention. The strip in FIG. 3 comprises a substrate 320, a first metallic conductor 332, a second metallic conductor 334, a first electrode 340, a second electrode 350, a first isolating layer 360, and second isolating layer 370. The first metallic conductor 332 and the second metallic conductor 334 are disposed on the substrate 320, whereas the first electrode 340 and the second electrode 350 are further disposed on the first metallic conductor 332 and the second metallic conductor 334 respectively. In addition, the first electrode 340 and the second electrode 350 are covered by the first isolating layer 360. The second isolating layer 370 may be further disposed on the first isolating layer 360, based on design requirements.

The substrate 320, the first isolating layer 360, and the second isolating layer 370 may be made of isolating materials, such as polyethylene terephthalate (PET). In contrast, the first metallic conductor 332 and the second metallic conductor 334 are made of metallic materials, such as silver. The first electrode 340 and the second electrode 350 are made of known materials for electrodes, such as carbon.

In some other embodiments, the numbers of the first metallic conductor 332, the second metallic conductor 334, the first isolating layer 360, and the second isolating layer 370 may be modified based on design considerations. For example, the first isolating layer 360 and the second isolating layer 370 in FIG. 3 are engraved with channels for blood samples. However, based on the design of channels, some strips may contain only the first isolating layer 360 in other embodiments. The strip in FIG. 3 comprises the first electrode 340 and the second electrode 350. However, in some embodiments the glucometer may use a three electrode design.

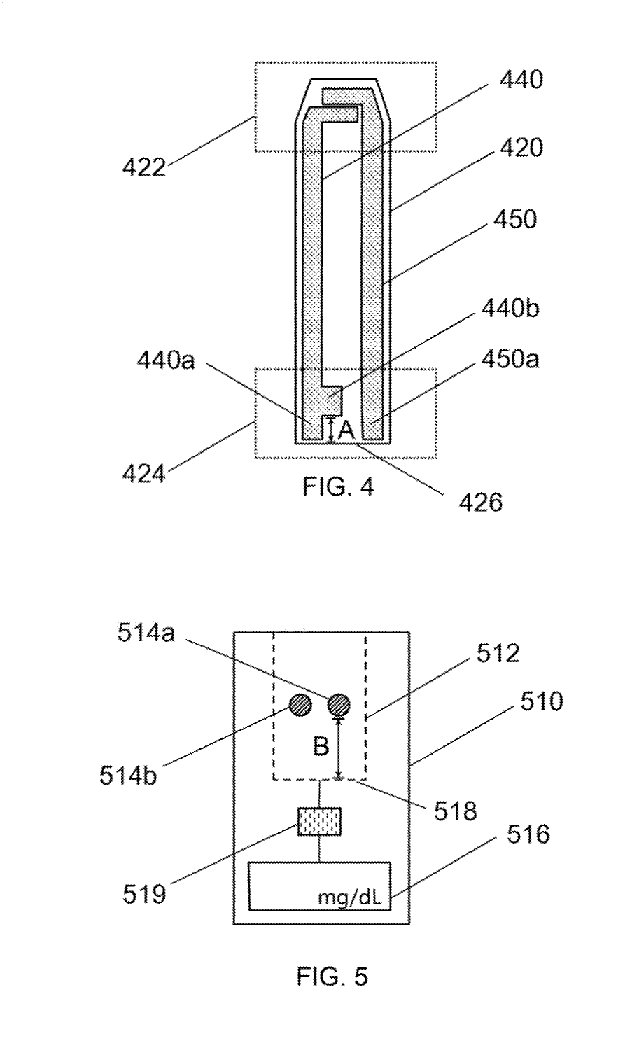

FIG. 4 is schematic views illustrating a strip, in accordance with one embodiment of the present invention. The strip in FIG. 4 comprises a substrate 420 as well as a first electrode 440 and a second electrode 450 which are disposed on the strip 420. Two regions on the substrate 420 are defined, based on their functions, as the sample end 422 and the connection end 424 respectively. The sample end 422 is configured to receive a blood sample while the connection end 424 is configured to connect with the slot on the glucometer. When the connection end 424 is inserted into the slot of a glucometer and a blood sample is provided onto the sample end 422, the blood sample electrically links the first electrode 440 and the second electrode 450, which were independent, and activate the glucometer to detect the blood glucose level of the blood sample on the sample end 422.

In FIG. 4, one margin of the substrate 320 in the connection end 424 is defined as a bottom edge 426. A first pin 440a, a second pin 440b, and a third pin 450a are protruded from the first electrode 440 and the second electrode 450 respectively to the bottom edge 426. However, the protrusion of the second pin 440b stops at a place a first distance A inward from the bottom edge 426. The first pin 440a and the second pin 440b are extensions of the first electrode 420 in the present embodiment. Therefore, the first pin 440a, the second 440b, and the first electrode 420 are made of the same material and electrically connected to each another.

FIG. 5 is schematic views illustrating a glucometer, in accordance with one embodiment of the present invention. The glucometer 510 in FIG. 5 comprises a slot 512, a display 516, and a sensor 519, in which the sensor 519 is electrically connected with the slot 512 and the display 516 respectively. The end of the slot 512 is defined as a bottom 518. The slot 512 comprises a first contact site 514a and a second contact site 514b parallel to the bottom 518. More specifically, both the first contact site 514a and the second contact site 514b are a second distance B away from the bottom 518. Once the sensor 519 detects that a short circuit formed between the first contact site 514a and the second contact site 514b, the sensor 519 activate the glucometer 510.

In some preferred embodiments, the second distance B is greater than the first distance A in light with FIGS. 4 and 5. In some other preferred embodiments, the second distance B is equal to the first distance A.

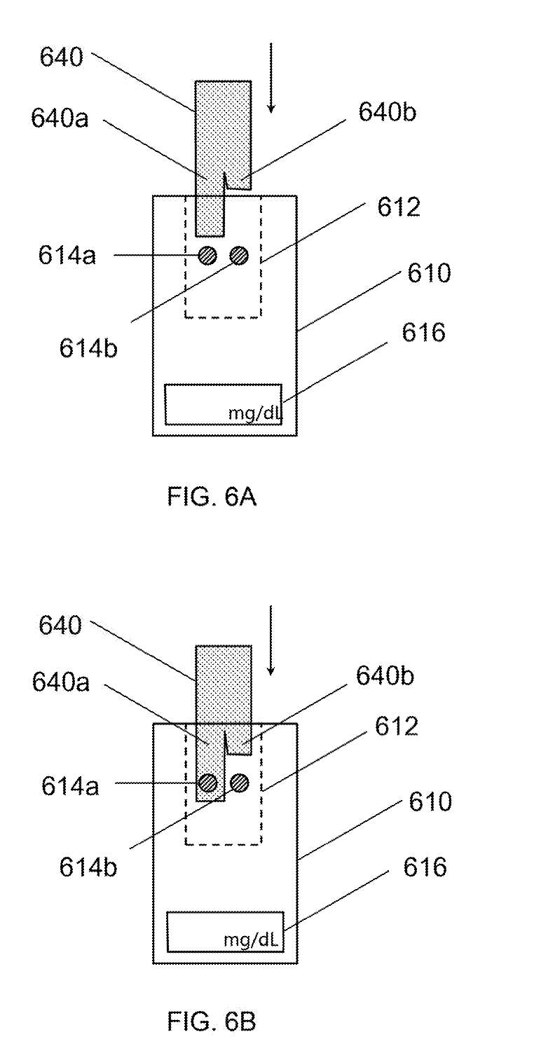

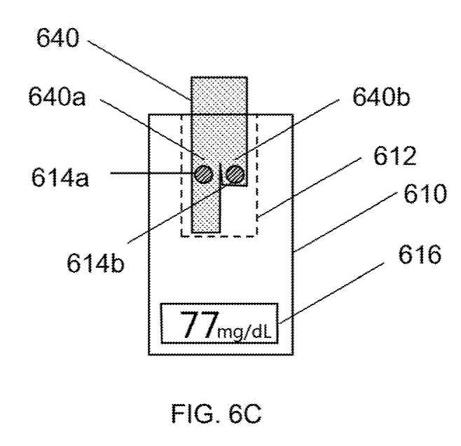

FIGS. 6A-6C are schematic views illustrating a system for activating glucometers, in accordance with one embodiment of the present invention. The system for activating glucometers comprises a glucometer 610 and a first electrode 640 disposed on a strip (not shown). The glucometer 610 further comprises a slot 612, a first contact site 614a, a second contact site 614b, and a display 516. If the strip is inserted into the slot 612, the first pin 640a and the second pin 640b of first electrode 640 are electrically connected to the first connection site 614a and the second connection site 614b respectively. The electrical connection results in a short circuit between the first contact site 614 and the second contact site 614b, and therefore triggers the activation of the glucometer 610.

More particularly, FIGS. 6A-6C illustrate a system for activating glucometers embodied by a glucometer 610 and the process of inserting a trip into the glucometer 610 for activation. FIG. 6A illustrates the early stage of the process that inserting the strip into the glucometer 610. In FIG. 6A, the first pin 640a and the second pin 640b are separated from the first contact site 614a and the second contact site 614b disposed in parallel. FIG. 6B illustrates the middle stage of the process, in which the strip is not fully inserted into the glucometer 610. In FIG. 1B, the first pin 640a has contacted with the first contact site 614a while the second pin 640b, the shorter pin, is still separated from the second contact site 614b. The glucometer 610 in FIG. 6B is still not activated and no blood glucose reading is presented on the display 616. FIG. 6C illustrates the late stage of the process, in which the strip is fully inserted into the slot 612. In FIG. 6C, the first pin 640a and the second pin 640b are contacting with the first contact site 614a and the second contact site 614b which are disposed in parallel. Therefore, the glucometer is activated and the blood glucose reading is presented on the display 616. Accordingly, the system for activating glucometers in FIGS. 6A-6C utilizes one shorten pin, amongst all, to ensure that the glucometer is activated to detect blood glucose levels when the strip is more in position.

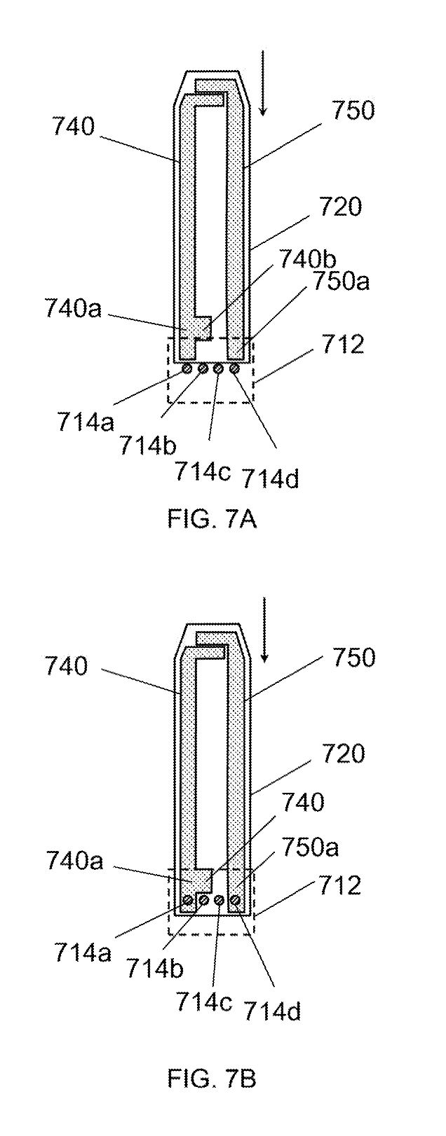

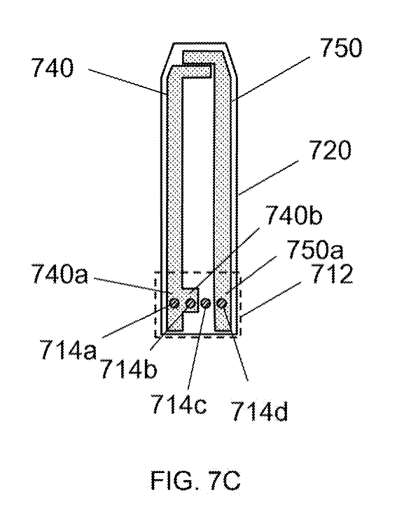

FIGS. 7A-7C are schematic views illustrating a system for activating glucometers, in accordance with one embodiment of the present invention. The system for activating glucometers comprises a strip and a slot 712 on a glucometer (not shown). The strip comprises a substrate 720 as well as a first electrode 740 and a second electrode 750 disposed on substrate 720. The slot 712 comprise a first contact site 714a, a second contact site 714b, a third contact site 714c, and a fourth contact site 714d which are disposed in parallel. As the strip is inserted into the slot 712, a first pin 740a and a second pin 740b protruding from the first electrode 740 are electrically connected with the first contact site 714a and the second contact site 714b to activate the glucometer; simultaneously, the third pin 750a extending from the second electrode 750 is electrically connected with the fourth contact site 214d to complete the detection of blood glucose levels.

More particularly, FIGS. 7A-7C illustrate a system for activating glucometers exemplified by the process of inserting a trip into the slot 712. FIG. 7A illustrates the early stage of the process that inserting the strip into the slot 712. In FIG. 7A, the first pin 740a, the second pin 740b, and the third pin 750a are separated from the first contact site 714a, the second contact site 714b, and the fourth contact site 714d which are disposed in parallel. The glucometer 110 in FIG. 7A is therefore not activated. FIG. 7B illustrates the middle stage of the process, in which the strip is not fully inserted into the slot 712. In FIG. 7B, the first pin 740a and the third pin 250a are both contacting with the first contact site 714a and the fourth contact site 214d disposed in parallel. However, since the second pin 740b is shorter than the others and is still separated from the second contact site 714b, the glucometer is remained inactivated. FIG. 7C illustrates the late stage of the process, in which the strip is fully inserted into the slot 712. In FIG. 7C, the first pin 740a, the second pin 740b, and the third pin 250a are contacting with the first contact site 714a, the second contact site 714b, and the fourth contact site 714d disposed in parallel. The short circuit between the first contact site 714a and the second contact site 714b therefore induces the activation of the glucometer and the electrical connection between the first contact site 714a and the fourth contact site 714d is used to detect the blood sample on the strip.

There are many inventions described and illustrated above. The present inventions are neither limited to any single aspect nor embodiment thereof, nor to any combinations and/or permutations of such aspects and/or embodiments. Moreover, each of the aspects of the present inventions, and/or embodiments thereof, may be employed alone or in combination with one or more of the other aspects of the present inventions and/or embodiments thereof. For the sake of brevity, many of those permutations and combinations will not be discussed separately herein.

What is claimed is:

1. A system for activating glucometers, comprising:
a strip, comprising:
a substrate, comprising:
a sample end; and
a connection end having a bottom edge; and
a first electrode configured on the strip, wherein the first electrode comprises:
a first pin, extending to the bottom edge; and
a second pin, extending to a place a first distance inward from the bottom edge, wherein the first pin is electrically connected with the second pin; and
a glucometer, comprising:
a slot, configured to receive the connection end, wherein the slot comprises:
a first contact site, configured to electrically connect with the first pin; and
a second contact site, configured to electrically connect with the second pin; and
a sensor, electrically connected to the slot and configured to activate the glucometer once a predetermined event is detected;
wherein the predetermined event is that the first pin and the second pin are electrically connecting to the first contact site and the second contact site respectively.

2. The system for activating glucometers as claimed in claim 1, wherein the system comprises a second electrode configured on the strip, and wherein the second electrode comprises a third pin extending to the bottom edge.

3. The system for activating glucometers as claimed in claim 2, wherein the system comprises:
a first metallic conductor, disposed between the substrate and the first electrode; and
a second metallic conductor, disposed between the substrate and the second electrode.

4. The system for activating glucometers as claimed in claim 2, wherein the system comprises an insulating layer disposed on the first electrode and the second electrode.

5. The system for activating glucometers as claimed in claim 1, wherein the slot has a bottom.

6. The system for activating glucometers as claimed in claim 5, wherein the first contact site and the second contact site are both a second distance away from the bottom.

7. The system for activating glucometers as claimed in claim 6, wherein the second distance is greater than or equal to the first distance.

8. The system for activating glucometers as claimed in claim 7, wherein the second distance is equal to the first distance.

9. The system for activating glucometers as claimed in claim 1, wherein the glucometer is an electrochemical glucometer.

10. The system for activating glucometers as claimed in claim 1, wherein the first contact site is also electrically connected to the second contact site in the predetermined event.
